(19) United States
(12) Patent Application Publication
(10) Pub. No.: US 2016/0362354 A1
(43) Pub. Date: Dec. 15, 2016

(54) METHOD FOR PRODUCING BIOFUELS FROM FRUIT KERNELS

(71) Applicant: Gerardo ULIBARRI, Ontario (CA)

(72) Inventor: Gerardo ULIBARRI, Ontario (CA)

(21) Appl. No.: 15/121,704

(22) PCT Filed: Feb. 25, 2015
(86) PCT No.: PCT/CA2015/050140

§ 371 Date: Aug. 25, 2016

(30) Foreign Application Priority Data
Feb. 26, 2014 (CA) 2 843 870

(51) Int. Cl. C07C 31/08; C07D 307/50; C12P 7/06; C10L 1/02

(52) U.S. Cl. CPC C07C 31/08; C10L 1/02; C07D 307/50; C12P 7/065; C10L 2290/26; C10L 2290/544; C10L 2290/28; C10L 2290/547; C10L 2290/08; C10L 2290/06; C10L 2200/0469

(57) ABSTRACT

Disclosed is the use of a fruit kernel for producing biofuel or an intermediate in the production thereof. The fruit kernel is generally obtained from an avocado or mango and is used as a dry powder. Also disclosed are methods for producing a biofuel from a fruit kernel or intermediates in the production thereof. These fruit kernels are of particular interest since they are a waste-product of the fruit. As such, the fruits can be grown for their normal purpose and the part of the fruit normally discarded as waste, namely the kernel, used to produce a biofuel.

### FIELD OF THE INVENTION

The present invention generally relates to bioprocessing. More specifically, the invention relates to a method for producing biofuels from fruit kernels, such as avocados and mangos.

### BACKGROUND OF THE INVENTION

With the new millennium came an increased understanding that biological material could be used to generate biofuels, such as bioethanol, and thus be used as a cleaner and more environmentally friendly additive and in some cases alternative to fossil fuel.

The robust nature of starchy grains, such as maize, cultivation throughout the world made it a prime candidate for being a source of biological material for biofuel production. However, large scale biofuel production requires a significant amount of starting material, thus putting stress on the grain supply chain for food consumption. In addition, the high cost of fossil fuel has resulted in suppliers looking to reduce costs by adding a percentage of bioethanol to the fossil fuel prior being sold at the pump. This has caused fuel companies to pay a higher price for grains, thus driving up the price of grains for food consumption.

Concerns have also been raised over whether the higher prices paid to farmers by fuel producers for grains will cause farmers that traditionally produce these grains for human consumption to consider biofuel applications for their produce. In the long run, this may negatively impact the amount of food grains that is available to the consumer for edible consumption.

Based on the foregoing, there is a need to find alternative sources for biofuel, such as bioethanol, production that limit the number of negative effects on the food supply chain.

### SUMMARY OF THE INVENTION

It is an object of the present invention to provide a method for producing biofuels from fruit kernels.

According to an aspect of the present invention, there is provided a method for producing an intermediate in the production of a biofuel from a fruit kernel. The method involving the steps of: grinding the fruit kernel to form a dry powder; dissolving the dry powder in an ionic liquid in the presence of a metal halide and an inorganic acid; and extracting the intermediate from the ionic liquid.

In one embodiment, the fruit kernel is obtained from an avocado or mango.

In another embodiment, the ionic liquid is 1-Octyl-3-methyl-1H-imidozol-3-ium chloride.

In a further embodiment, the metal halide, inorganic acid and solvent is CrCl₂, hydrogen chloride, and ethyl acetate.

In a still further embodiment, the intermediate is hydroxymethyl furfural.

According to an aspect of the present invention, there is provided a method for producing biofuels from fruit kernels. The method comprising the steps of: grinding the fruit kernel under a solution of inhibitor such as bisulfite (e.g. NaHSO₃) or metabisulfite (e.g. Na₂S₂O₇) or directly exposed to the air; heating the ground fruit kernel under reflux conditions in an alcohol; filtering the residual solid from the heating step; drying the filtered residual solid; mixing the filtered residual solid with an acid solution (preferably 5-10%) or an enzymatic hydrolytic process; baking the filtered residual solid and acid mixture; filtering the baked residual solid; and fermenting the filtrate to produce an organic fuel.

In one embodiment, the fruit kernels are from avocados or mangos or another starchy fruit kernel.

In another embodiment, the alcohol used in the heating step is methanol or ethanol, pure or diluted.

In a further embodiment, the step of drying the filtered solution takes place at room temperature, in an oven and/or under vacuum.

In a still further embodiment, the above acid solution is a sulfuric acid solution.

In a yet further embodiment, the baking step takes place in an autoclave under pressure or at atmospheric pressure.

In another embodiment, the temperature of the autoclave is between about 100° C. and about 140° C. In some cases, the baking step is for about 30 mins to about 1 hour.

In a further still embodiment, the method further comprises cooling the baked residual solid prior to filtering.

In an embodiment, the filtrate is neutralized (pH between 6 and 8) using a strong base, preferably diluted NaOH solution.

In an embodiment, the filtrate is fermented in the presence of Clostridium acetobutylicum, ATCC 824 or Saccharomyces cerevisiae.

According to another aspect of the present invention, there is provided use of fruit kernels for production of biofuels. Preferably, the fruit kernels are from avocados or mangos. The fruit kernels being in the form of dried powder.

According to another aspect of the present invention, there is provided a biofuel produced from a fruit kernel. The fruit kernel being obtained from an avocado or mango.

### DESCRIPTION OF THE INVENTION

The following description is of one particular embodiment by way of example only and without limitation to the combination of features necessary for carrying the invention into effect.

The method described herein is for producing biofuels, such as bioethanol and bioalcohols, from fruit kernels. In this case, the term bioalcohol refers to any alcohol, such as ethanol or butanol, that is produced from a biological source. The method uses fruit kernels, also known as pits or seeds, which are high in starch content to produce biofuels. Preferred fruit kernels for the purpose of the present method, include, but are not limited to, avocados and mangos. These fruit kernels are of particular interest since they are a waste-product of the fruit. As such, the fruits can be grown for their normal purpose and the part of the fruit normally discarded as waste, namely the kernel, used to produce a biofuel.

For the purpose of this discussion, the method will be described with reference to avocado kernels as the starting material. As mentioned above, it should be readily understood that the method is applicable to any fruit kernel having a high starch content.

The avocado kernels are ground down to a fine, medium or course grind using any one of a number of different methods that are known in the art, such as, mechanical pulverization using a waring blender or the like. The kernels can be freshly extracted from the fruit, or can be dried. If the kernel is obtained from a fresh fruit, the kernel should be ground into a fine paste or grated into slivers, under a solution of inhibitor such as bisulfate (e.g. NaHSO₃) or metabisulfite (e.g. Na₂S₂O₇) or directly exposed to the air, and air dried for at least 24 hours.

The dried kernels in powder form can be used directly to obtain hydroxymethyl furfural (HMF), which can be used as a starting material for the syntheses of various carbon compounds including liquid biofuels, such as dimethylfuran (DMF), liquid alkanes, thermo-resistant polymers and complex macrocycles (Lee J-W et al., Carbohydrate Research 346:177-182, 2011). The dry kernel powder is dissolved in an ionic liquid in the presence of a metal halide and an inorganic acid to produce HMF. The HMF is then extracted using known techniques, such as organic solvent extraction or using chromatographic techniques (see Chun J-A et al., Starch/Starke 62: 326-330, 2010, the contents of which are incorporated herein by reference).

In another embodiment, the ground kernels are then mixed with an alcohol, such as methanol or ethanol, and heated under reflux conditions for a period of time, typically 6 to 24 hours. A Sohxlet apparatus can also be used to extract soluble components of the kernel. Following heating of the ground kernels, the residual solid leftover from the reflux reaction is filtered, to remove the remaining alcohol. The filtered solid is then dried at room temperature or in an oven at a temperature suitable for removing liquid from the mixture, without cooking the solid. Alternatively, when the residual solid is filtered, the filtering can take place using a vacuum to improve the efficiency of liquid removal from the residual solid. Moreover, a vacuum can be used separate from filtering the residual solid to dry the solid.

The dried solid is then mixed with an acid solution, such as sulfuric acid, and heated. In one embodiment, the acid solution is a 5-10% (v/v) acid solution. Preferably, an autoclave is used to heat the mixture. For example, an autoclave set at between 100-140° C. under pressure or at atmospheric pressure is used and the mixture baked for about 30 minutes to about 1 hour. Afterwards the mixture is cooled and then filtered.

The resulting neutralized filtrate contains approximately 40 mg/ml sugars, expressed as glucose, which is produced from the hydrolysis of starch. This represents approximately 30-40% of the weight of the kernel. The resulting filtrate can then be fermented using the acetone-butanol-ethanol (ABE) fermentation process, which produces acetone, n-butanol and ethanol, or another suitable fermentation process. Suitable microorganisms for fermentation include: Clostridium acetobutylicum, ATCC 824 or Saccharomyces cerevisiae. These end products can be further refined into one or more biofuels.

It will be understood that numerous modifications thereto will appear to those skilled in the art. Accordingly, the above description should be taken as illustrative of the invention and not in a limiting sense. It will be further understood that it is intended to cover any variations, uses, or adaptations of the invention following, in general, the principles of the invention and including such departures from the present disclosure as come within known or customary practice within the art to which the invention pertains and as may be applied to the essential features herein before set forth, and as follows in the scope of the appended claims.

### EXAMPLE

Hydroxymethylfurfural (HMF) was produced from avocado kernels. Previously dried avocado kernels and avocado kernels obtained from fresh fruit were used as the starting material. In the case of fresh kernels, the kernels were removed from the fruit and ground into a fine paste and air-dried for at least 24 hours before grinding into a fine powder using a mill. Previously dried avocado kernels were also ground to a fine powder using a mill.

1-Octyl-3-methyl-1H-imidozol-3-ium chloride [OMIM]Cl (4g), CrCl₂ (0.2 g) and ethyl acetate (1 ml) were placed in a flask and heated to reflux of ethyl acetate for 10 mins in an oil bath with an external temperature of ˜120° C.

After pre-heating, a suspension of 1 gram of dried kernel powder and HCl (5 ml) were added to the above solution. This mixture was brought to reflux for at least 3 hours or until complete production of HMF was achieved as determined by thin layer chromatography or gas chromatography-mass spectrometry.

The solution was allowed to cool to room temperature and the HMF was extracted several times using the ethyl acetate solution. The extracts were evaporated to recover 0.209 g of HMF.

1.-3. (canceled)

4. A method for producing an intermediate in the production of a biofuel from a fruit kernel, comprising the steps of:
grinding the fruit kernel to form a dry powder;
dissolving the dry powder in an ionic liquid in the presence of a metal halide and an inorganic acid; and
extracting the intermediate from the ionic liquid.

5. The method of claim 4, wherein the fruit kernel is obtained from an avocado or mango.

6. The method of claim 4, wherein the ionic liquid is 1-Octyl-3-methyl-1H-imidozol-3-ium chloride.

7. The method of claim 4, wherein the metal halide and inorganic acid and solvent is CrCl₂, hydrogen chloride and ethyl acetate.

8. The method of claim 4, wherein the intermediate is hydroxymethyl furfural.

9. A method for producing biofuels from fruit kernels comprising the steps of:
grinding the fruit kernel;
heating the ground fruit kernel under reflux conditions in an alcohol;
filtering the residual solid from the heating step;
drying the filtered residual solid;
mixing the filtered residual solid with an acid solution;
baking the filtered residual solid and acid mixture;
filtering the baked residual solid; and
fermenting the filtrate to produce an organic fuel.

10. The method of claim 9, wherein the fruit kernels are from avocados or mangos.

11. The method of claim 9, wherein the alcohol used in the heating step is methanol or ethanol.

12. The method of claim 9, wherein the step of drying the filtered solution takes place at room temperature, in an oven and/or under vacuum.

13. The method of claim 9, wherein the acid solution is a sulfuric acid solution.

14. The method of claim 9, wherein the baking step takes place in an autoclave under pressure or at atmospheric pressure.

15. The method of claim 14, wherein the temperature of the autoclave is between about 100° C. and about 140° C.

16. The method of claim 14, wherein the baking step is for about 30 mins to about 1 hour.

17. The method of claim 9, further comprising cooling the baked residual solid prior to filtering.

18. The method of claim 9, further comprising neutralization of the filtrate.

19. The method of claim 9, wherein the filtrate is fermented in the presence of Clostridium acetobutylicum, ATCC 824 or Saccharomyces cerevisiae.

20. The method of claim 9, wherein the acid solution is a 5 to 10% (v/v) acid solution.

21. A biofuel produced from a fruit kernel.

22. The biofuel of claim 21, wherein the fruit kernel is obtained from an avocado or mango.
